(19) United States
(12) Patent Application Publication
(10) Pub. No.: US 2024/0415111 A1
(43) Pub. Date: Dec. 19, 2024

(54) SYSTEM FOR SUPPORTING BIOLOGICALLY ENGINEERED ORGANS

(71) Applicant: Miromatrix Medical Inc., Eden Prairie, MN (US)

(72) Inventors: Aleksandr Katane, Victoria, MI (US); Aron Stumbras, Eden Prairie, MN (US); Dmitrii Pokhill, Eden Prairie, MN (US); Shawn Riesgraf, Carver, MN (US)

(21) Appl. No.: 18/702,784

(22) PCT Filed: Oct. 18, 2021
(86) PCT No.: PCT/US2022/078287

§ 371 Date: Apr. 18, 2024

Related U.S. Application Data
(60) Provisional application No. 63/256,981, filed on Oct. 18, 2021.

(51) Int. Cl. A01N 1/02

(52) U.S. Cl. CPC A01N 1/0247

(57) ABSTRACT

A system (200) for growing or supporting an organ (50), in particular a biologically engineered organ, comprises: an enclosure (202); a perfusate circuit: a perfusate pump (206); a gas transfer unit (208) and a optionally a gas mixture unit connected to the gas transfer unit; one or more sensors (212a, 212b); an injection system (216) configured to inject nutrients; and a controller (214). The controller can be configured to operate the pump, the gas transfer unit, and the injection system based on sensor signals from the sensor(s) to grow or support the organ in an automated or semi-automated fashion.

### CLAIM OF PRIORITY

This patent application claims the benefit of priority, under 35 U.S.C. Section 119 (e), to Aleksandr Katane, U.S. Patent Application Ser. No. 63/256,981, entitled “ASSEMBLING AND SUPPORTING BIOLOGICALLY ENGINEERED ORGANS,” filed on Oct. 18, 2021, which is hereby incorporated by reference herein in its entirety.

### BACKGROUND

Organ transplants are common across the United States and throughout the world. Organs such as livers, kidneys, pancreas, lungs and hearts are commonly transplanted to prolong the life of the recipients. However, there is often a shortage of organs, creating a demand and a waiting list for the organs. One solution to this shortage is to use biologically engineered organs and alternative tissues, where organs are stripped of their cells and the recipient's cells can be perfused into the biologically engineered organ, to help prevent rejection.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 illustrates an overview of devices and systems involved in stages of transplanting a biologically engineered organ.

FIG. 2 illustrates a schematic view of a system for growth and support of a bio-engineered organ.

FIG. 3 illustrates a schematic view of a system for growth and support of a bio-engineered organ.

FIG. 4A illustrates a schematic view of a system for growth and support of a bio-engineered organ.

FIG. 4B illustrates a schematic view of a system for growth and support of a bio-engineered organ.

FIG. 5 illustrates a schematic view of a method of operating a system for growth and support of a bio-engineered organ.

FIG. 6 illustrates a block diagram of architecture for an example computing system used.

FIG. 7 illustrates a schematic view of a system for growth and support of a bio-engineered organ.

FIG. 8 illustrates a schematic view of a system for growth and support of a bio-engineered organ.

### DETAILED DESCRIPTION

Biologically engineered organs (BEOs) and advanced bio-engineered tissues (ATs) can help to recapitulate native organ function while helping to reduce rejection, and helping to reduce organ wait times. BEOs can also help to reduce wait times by making use of organs of different species. In either scenario, the BEO is typically processed at a laboratory under tightly controlled conditions before it can be transported to a hospital for implantation. For example, ATs and BEOs should be conditioned with appropriate biochemical and physical cues to promote development and maintenance of physiological function during growth and support of the AT or BEO. BEOs can include livers, lungs, hearts, pancreases, kidney, intestine, or the like, and each type of organ can require careful monitoring and control during growth and support of the AT or BEO. However, conditioning an organ can be a very time-consuming process requiring significant manual labor. This disclosure discusses systems that can help address these issues by using a system that can accept an AT or BEO and can connect to the tissue's vascular system. The system can perfuse the AT or BEO with media or blood, when necessary. Once the AT or BEO has established active flow, the system can be used to test various function parameters. These functional tests can be specific to the AT or BEO that is installed into the system. The system can further determine, based on tests or sensor data, requirements of the AT or BEO for optimal growth or support of the AT or BEO.

For example, while the AT or BEO is being perfused, the system can be dosed with various chemicals, drugs, and/or proteins to challenge the AT or BEO for function based on the measured parameters. Samples of the media or blood can be collected and can be analyzed to determine functional adequacy. The system can also actively monitor the housed AT or BEO for viability and function to ensure that it meets specifications prior to implantation. By automatically adjusting operation of the system or automatically injecting nutrients to the perfusate, the system can help to reduce labor requirements for growing or maintaining a BEO or AT prior to transport and transplantation. And by automatically compensating for nutrient depletion based on sensor feedback, the system can also reduce supply chain inefficiency by helping to reduce unnecessary consumption/replenishment of perfusate/media.

The above discussion is intended to provide an overview of subject matter of the present patent application. It is not intended to provide an exclusive or exhaustive explanation of the invention. The description below is included to provide further information about the present patent application.

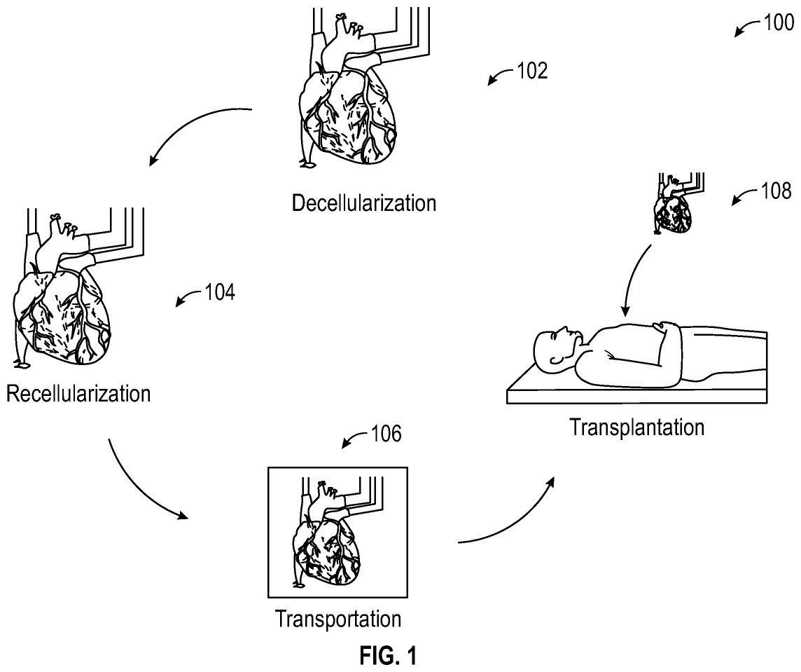

FIG. 1 illustrates an overview of devices and systems involved in stages of transplanting a BEO in accordance with at least one example of the present disclosure.

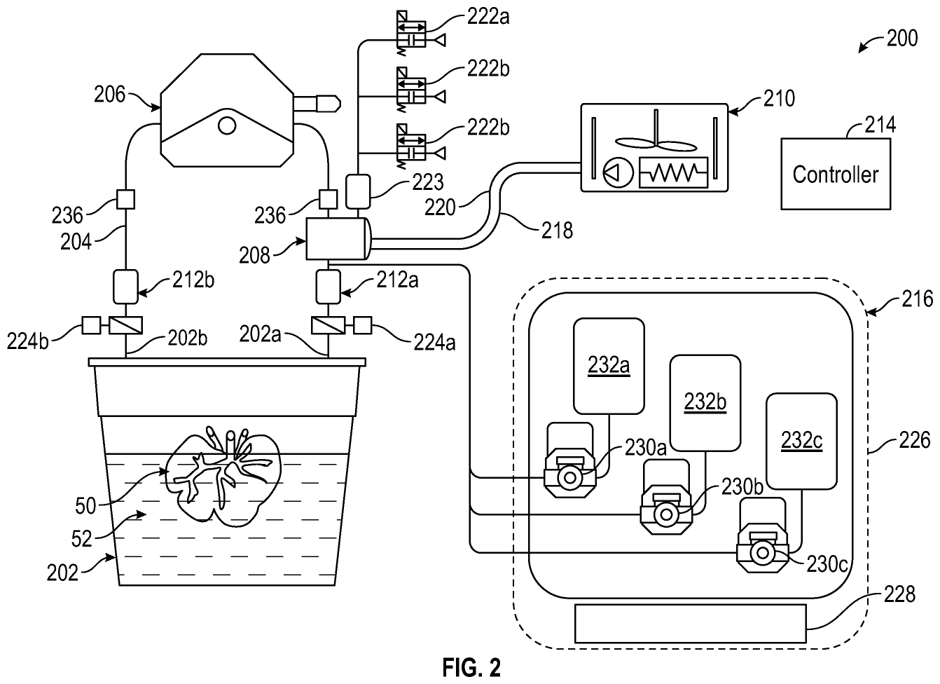

An example procedure 100 shows various stages that can be included in a transplantation procedure. At a stage 102, an organ can be received from a donor human or other species, such as pig, sheep, bovine, or the like. The organ can be decellularized in a laboratory under carefully controlled conditions, where all (or substantially all) cellular material can be removed from the organ. Following decellularization, the organ can be recellularized at a stage 104, whereby cells from the donor (or other sources) can be added to the organ and the organ can be functionally tested and otherwise prepared for implantation. Once it is determined that the BEO is ready for transplant, the BEO can be prepared for transportation at stage 106. Then, at step 108, the BEO can be implanted into the recipient in a transplant operation. Further details of several of these steps are discussed in further detail below with respect to FIGS. 2-6.

FIG. 2 illustrates a schematic view of a system 200 for functional testing, maintenance, or growth of a bio-engineered organ (BEO). The system 200 can include an enclosure 202, a perfusate circuit 204, a pump 206, a gas transfer system 208, a heating/cooling system 210, an inlet sensor suite 212a, an outlet sensor suite 212b, a controller 214, and an injection system 216. Also shown in FIG. 2 is an organ 50, which can be a liver, for example, and perfusate 52, which can be a fluid.

The enclosure 202 can be an enclosure configured to support the organ 50 therein in a perfusate flow. The enclosure 202 can include a perfusate inlet 202a and a perfusate outlet 202b to receive the perfusate flow through the enclosure and through the organ 50. The enclosure 202 can be a rigid or semi-rigid container, enclosure, or housing made of materials such as one or more of metals, plastics, foams, elastomers, ceramics, composites, combinations thereof, or the like, such as polycarbonate. The enclosure 202 can be transparent or translucent to provide visibility of the organ 50 therein for operators or technicians. Optionally, the enclosure 202 can be configured to limit exposure of the organ 50 to light. The enclosure 202 can include one or more latches, fasteners, or the like to resealably close a lid in an air-tight manner. In some examples, the enclosure 202 can include one or more hinges. The enclosure 202 can include one or more insulative layers to help thermally isolate contents of the enclosure 202 from ambient conditions. The enclosure 202 can include a seal between the lid and the container of the enclosure 202.

The enclosure 202 can be configured to receive and support a biologically engineered organ therein in a perfusate flow and/or bath. That is, the enclosure 202 can be shaped to support an organ therein. In some examples, the enclosure 202 can be organ-specific (such as kidney specific) such that the enclosure 202 is shaped to support the kidney (or other organ in other examples). In some examples, the organ-specific enclosure 202 can be removable and replaceable as required for an organ to be transported. The perfusate inlet 202a and the perfusate outlet 202b can include sterile-quick connects for disconnecting the enclosure 202 from the perfusate circuit 204.

The perfusate circuit 204 can be connected to the perfusate inlet 202a and the perfusate outlet 202b and can be configured to transmit perfusate through the system and its components. The circuit 204 can be made of one or more types of tubing including rigid, semi-rigid, and flexible tubing on various materials (e.g., copper, steel, aluminum, plastics, silicone, or the like). Optionally, the tubing of the circuit 204 can be low-shed tubing or pharmacy-grade tubing. Additionally, the perfusate circuit 204 can be configured to have relatively few restriction points throughout the perfusate circuit 204 to help reduce pressure drop through the perfusate circuit 204, which helps increase an accuracy between pressure measured outside the organ 50 and at the pressure sensor (e.g., the inlet sensor suite 212a or the outlet sensor suite 212b) and pressure at or within the organ 50.

The tubing or circuit 204 can optionally connect directly to perfusion vessels of the organ 50, which can allow for independent vessel interfacing. Though perfusate is discussed generally with respect to FIG. 2, the perfusate circuit can be configured to handle one or more other fluids, such as media or blood. In some examples, patient-specific donor blood can be used in the circuit 204, which can help to qualify patient specific compatibility.

The pump 206 can be connected to the circuit 204 and can be configured to circulate perfusate through the perfusate circuit 204. The pump 206 can be a positive displacement pump, a centrifugal pump, or an axial pump. The pump 206 can be a low-shear pump to help limit damage to the fluid and can optionally be reversible. In some examples, the pump 206 can be a continuous type pump or a peristaltic type pump (with or without damping). In some examples, the pump 206 can be located downstream of the outlet 202b of the enclosure 202 and upstream of the gas transfer unit 208.

The gas transfer unit 208 can be connected to the perfusate circuit 204 and configured to transfer gas to and from the perfusate, such as oxygen, nitrogen, argon, and carbon dioxide (CO2). The gas transfer unit 208 can be either an active gas transfer unit or a passive gas transfer unit configured to maintain oxygen and CO2 at a desired concentration range within the perfusate. In some examples, the gas transfer unit 208 can manage other gasses conducive to organ culturing. The gas transfer unit 208 can be located downstream of the pump 206 and upstream of the inlet sensors 212. The gas/transfer unit 208 can optionally include a heat exchanger configured to exchange heat between perfusate of the perfusate circuit 204 and the heating and cooling system 210. Optionally, a separate heat exchange can be used in the system 200. The gas transfer unit 208 can also optionally include an air separator or a bubble trap. Optionally, the gas transfer unit 208 can be an oxygenator.

The heating/cooling system 210 can be connected to the perfusate circuit 204 to exchange heat with the perfusate such that the perfusate is delivered to the enclosure 202 and to the organ 50 within a target temperature range. The heating/cooling system 210 can be connected to the gas transfer unit 208 such as to allow the heat exchanger of the gas transfer unit 208 to exchange heat with the perfusate in the perfusate circuit 204. For example, a supply line 218 and a return line 220 can connect the heating/cooling system 210 to the gas transfer unit 208, such as to the heat exchanger of the gas transfer unit 208.

The heating/cooling system 210 can be a heating system in some examples and can be a cooling system in some examples. In some examples, the system 210 can include discrete heating and cooling systems. For example, the system 210 can include a resistive heater, fan, and heat exchanger. In other examples, the system 210 can be, for example, a refrigerant heat pump including a compressor, a condenser, an evaporator, one or more expansion valves, and a reversing valve. In some examples the system 210 can be a thermoelectric heating and cooling device (Peltier device). In some examples, the system can include a refrigeration cooling system and a resistive electric heating device. In any of these examples, the system 210 can include a heat exchanger configured to exchange heat with the perfusate indirectly. Optionally, the system 210 can include an emergency cooling system.

Optionally, the gas transfer unit 208 can include or can be connected to one or more gas mixture units 222a-222n. The gas mixture units 222 can each be connected to the gas transfer unit 208 and can be configured to deliver gas to the gas transfer unit 208. The gas mixture units 222 can each be configured to deliver one or more of Carbon Dioxide, Dioxygen, Nitrogen, or Argon to the gas transfer unit 208. Optionally, gas blends can be delivered such as O2 at a blend of 20% O2 to air to 80% O2 to air.

The gas mixture units 222 can each include a gas storage unit, a control valve, or a gas meter. Optionally, a single mass/flow controller 223 can be connected to the gas mixture units 222 upstream of the gas transfer unit 208. The flow controller 223 can optionally include a gas meter or flow meter and one or more control valves. The flow meter and the control valves of the flow controller 223 can be in communication with the controller 214. Each gas storage unit can be a tank, vessel, or the like configured to store a gas or fluid. The gas storage units of the gas mixture units 222 can be configured to supply the gas transfer unit 208 with gas (such as oxygen) for transfer to the perfusate. The control valves of the gas mixture units 222 can be in communication with a controller and can be operated to control flow of gas from the gas mixture units 222 to the gas transfer unit 208. The gas meters can each be configured to detect a flow of gas through each of the mixture units 222a-222n and to produce a signal based on the detected flow. Optionally, a pressure transducer or switch can be connected to each of the mixture units 222a-222n to limit gas pressure delivered by each of the mixture units 222a-222n to the gas transfer unit 208.

The system 200 can optionally include or can be connected to a power source that can be configured to power the pump 206, the heating/cooling system 210, the gas transfer unit 208, the various sensors of the system 200, the controller, a user interface, and/or other components of the system 200. The power source can be optionally uninterruptable, such as to provide continuous power to the system 200, even in the event of a loss of input power.

The inlet sensor suite 212a can optionally include one or more of a pressure sensor, a temperature sensor, a flow meter, a glucose sensor, an O2 sensor, a CO2 sensor, or other sensors. Each sensor can be connected to the perfusate circuit 204 upstream of the enclosure 202 (and can be optionally be within the enclosure 202). Similarly, the outlet sensor suite 212b can optionally include one or more of a pressure sensor, a temperature sensor, a flow meter, a glucose sensor, an O2 sensor, a CO2 sensor, an organ health sensor, or other sensors. Each sensor can be connected to the perfusate circuit 204 upstream of the enclosure 202 (and can be optionally be within the enclosure 202). Though the sensors are shown in FIG. 2 as having specific locations, the sensors can be positioned in various locations in other examples.

For example, a temperature sensor can be located upstream of the enclosure 202 or downstream of the enclosure 202. The inlet temperature sensor can be configured to produce an inlet temperature signal based on an inlet temperature of the perfusate entering the enclosure 202. The outlet temperature sensor can be configured to produce an outlet temperature signal based on an outlet temperature of the perfusate leaving the enclosure 202. The temperature sensor(s) be any type of fluid temperature sensor, either in a thermowell, coupled to a pipe of the circuit 204, or in direct contact with the process fluid, such as a thermistor, thermocouple, resistance temperature detector, or the like.

Various sensor options are discussed below. An inlet glucose sensor can be connected to the perfusate circuit 204, such as upstream of the enclosure 202. Similarly, an outlet glucose sensor can be connected to the perfusate circuit 204, such as downstream of the enclosure 202. The glucose sensor can be configured to produce a glucose sensor signal based on a glucose level of the perfusate. Similarly, an organ health sensor can be connected to the perfusate circuit 204, such as upstream or downstream of the enclosure 202. The organ health sensor can be configured to produce an organ health sensor signal based on a metabolite level of the perfusate within the circuit 204. An inlet CO2 sensor can be connected to the perfusate circuit 204 upstream of the enclosure 202. An outlet CO2 sensor can be connected to the perfusate circuit 204 downstream of the enclosure 202. Each of the inlet CO2 sensor and the outlet CO2 sensor can be a carbon dioxide sensor configured to produce a carbon dioxide signal based on a respective CO2 level of the perfusate of the perfusate. An inlet O2 sensor can be connected to the perfusate circuit 204 upstream of the enclosure 202. An outlet O2 sensor can be connected to the perfusate circuit 204 downstream of the enclosure 202. Each of the inlet O2 sensor and the O2 sensor can be an oxygen sensor configured to produce an oxygen signal based on a respective O2 level of the perfusate. An inlet pressure sensor can be connected to the perfusate circuit 204 upstream of the enclosure 202 and can be configured to produce an inlet pressure signal based on a pressure of the perfusate leading into the enclosure 202. An outlet pressure sensor can be connected to the perfusate circuit 204 upstream of the enclosure 202 and can be configured to produce an inlet pressure signal based on a pressure of the perfusate leading into the enclosure 202. Any or all of the sensor signals can be transmitted to the controller 214.

The perfusate circuit 204 can optionally include an inlet control valve 224a and an outlet control valve 224b. The inlet control valve 224a and the outlet control valve 224b can each be modulating valves or isolation valves in communication with the controller and can be operated thereby, such as to isolate the enclosure 202 and the organ 50, such as for removal of the enclosure 202 and the organ 50 from the system 200. Optionally the inlet control valve 224a and the outlet control valve 224b can be used to regulate flow through the enclosure 202 and the organ 50.

The sampling port 235 can be connected to the perfusate circuit 204 (for example downstream of the pump 206) and can be configured to receive a syringe to withdraw a sample of the perfusate without compromising sterility of the perfusate within the circuit 204. Similarly, the dosing port 236 can be connected to the perfusate circuit 204 (for example upstream of the pump 206) and can be configured to receive supplements for delivery to the perfusate and ultimately the organ without compromising sterility of the perfusate within the circuit 204.

The controller 214 can be a programable controller, such as a single or multi-board computer, a direct digital controller (DDC), a programable logic controller (PLC), or the like. In other examples the controller 214 can be any computing device, such as a handheld computer, for example, a smart phone, a tablet, a laptop, a desktop computer, or any other computing device including a processor, memory, and communication capabilities.

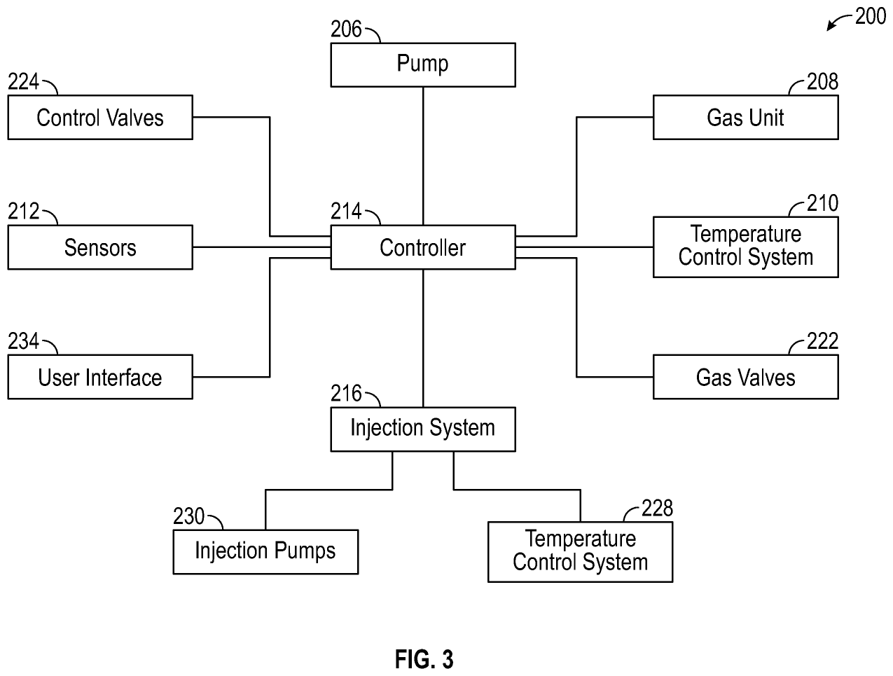

The controller 214 can generally be configured to control operations of the systems 200, such as by controlling operation of the pump 206, the gas transfer unit 208, the heating/cooling system 210, the power source, any sensor of the sensor suites 212, or the injection system 216. Various examples of how the controller 214 can control one or more components the system 200 to support or grow the organ 50 are discussed below with reference to FIG. 3.

The injection system 216 can include a housing 226, a heating/cooling system 228, pumps 230a-230c, and tanks 232a-232c. The injection system 216 can be configured to inject one or more nutrients or fluids into the perfusate circuit 204. The discharge of each pump 230 can be connected or manifolded together and can be connected to the perfusate circuit 204, such as upstream of the inlet sensor suite 212a and therefore the inlet control valve 224a and the perfusate inlet 202a. Optionally, the injection system 216 can connect to the perfusate circuit 204 upstream of the gas transfer unit 208 or in another location.

The enclosure 226 can include one or more walls and insulation, such as to provide a temperature-controlled environment within the enclosure 226. The enclosure 226 can made of materials such as one or more of metals, plastics, foams, elastomers, ceramics, composites, combinations thereof, or the like. The enclosure 226 can be transparent or translucent (or can include a transparent or translucent portion, e.g., window) to provide visibility of the pumps 230 and tanks 232. Optionally, the enclosure 226 can be configured to limit exposure of the tanks 232 to light. The enclosure 226 can include one or more latches, fasteners, or the like to resealably close a door or lid. In some examples, the enclosure 226 can include one or more hinges to connect a door. The enclosure 226 can include a seal between a door and the container of the enclosure 226.

The pumps 230 can each be configured to pump fluid therethrough and to discharge fluid to the perfusate circuit 204. Each of the pumps 230 can be any of the pump types discussed above with respect to the pumps 206. The tanks 232 can each be a storage container comprised of one or more of metals, plastics, foams, elastomers, ceramics, composites, combinations thereof, or the like. The tanks 232 can each be configured to store fluids to be injected into the perfusate circuit 204 as needed, or as determined by an operator or the controller 214. Though three tanks and pumps are shown the injection system 216 can include 1, 2, 4, 5, 6, 7, 8, 9, 10, or the like tanks or pumps.

The tanks 232 can be configured to store and the pumps 230 can be delivered to pump one or more of cell culture media, cells, glutamine, glucose, buffer, sodium chloride, essential amino acids, non-essential amino acids, drugs, ammonia (ammonium chloride), HEPES (C₈H₁₈N₂O₄S), Sodium Bicarbonate, Insulin, Epinephrine, Albumin, linoleic acid, dexamethasone, or glucagon. The drugs can be one or more of (R)-Warfarin, (S)-Mephenytoin, Acetaminophen, Aprepitant, Azole, Benzodiazepines, Beta, Caffeine, Calcium, Carbamazepine, Celecoxib, Clarithromycin, Codeine, Cyclosporine, Delavirdine, Dextromethorphan, Diazepam, Diclofenac, Enalapril, Erythromycin, Estradiol, Estrogen, Fentanyl, Finasteride, Flecainide, Fluoxetine, Glipizide, Glyburide, Haloperidol, Indinavir, Indomethacin, Lidocaine, Lopinavir, Loratidine, Methadone, Mexiletine, Morphine, Nelfinavir, Nifedipine, Olanzapine, Omeprazole, Opioid, Pentamidine, Phenothiazines, Phenytoin, Piroxicam, Prednisone, Progesterone, Propranolol, Quinidine, Risperidone, Ritonavir, Selective serotonin reuptake inhibitors, Saquinavir, Sildenafil, Sirolimus, Statins, Tacrine, Tacrolimus, Tamoxifen, Testosterone, Theophylline, Tramadol, Trazodone, Tricyclic, Valproate, Venlafaxine, Verapamil, or Voriconazole.

The injection system 216 can be controlled, such as by the controller 214, to inject one or more fluids into the perfusate circuit 204 such as to treat, grow, or support the organ 50 in the enclosure 202. Additional details of operation of the system 200 and the injection system 216 are discussed below with respect to FIG. 3.

FIG. 3 illustrates a schematic view of the system 200 for growth and support of a bio-engineered organ. The system 200 can be the same or similar to the system 200 discussed above with respect to FIG. 2; FIG. 3 shows how various components can be connected to the controller 214.

The pump 206, the gas transfer unit 208, the cooling system 210, the inlet sensor suite 212a, the outlet sensor suite 212b, the injection system 216, the gas mixture units 222, and the control valves 224 can be connected to the controller. The cooling system 228 and the pumps 230 can be connected directly the controller 214 or can be connected to the controller 214 through a controller or other device of the injection system 216.

The user interface 234 can be any display and/or input device. For example, user interface can be a touch screen display, computer, tablet, phone, or the like. In another example, user interface 234 can provide lights, buttons, and/or switches. The user interface 234 can be in communication with the controller 214 and configured to operate the controller 214 and devices connected thereto.

One or more of the various sensors and signals of the system 200 can be used by the controller 214 to grow or support the organ 50 within the system in an automated or semi-automated fashion, such as to help reduce time and labor required to grow or support the organ 50. The controller 214 can use various algorithms (e.g., PID loops) incorporating data from one or more sensors to perform such growth or maintenance of the organ. Various examples are discussed in further detail below.

In one example, the controller 214 can be configured to receive an inlet temperature signal from the inlet sensor suite 212a or the outlet temperature signal from the outlet sensor suite 212b. The controller 214 can further be configured to operate the pump 206, the gas transfer unit 208, or the heating and cooling system 210 based on the inlet temperature signal and the outlet temperature signal. For example, the controller 214 can activate the cooling of the system 210 when the outlet temperature signal indicates that a temperature of the perfusate in the circuit 204 is above a threshold, for example 37, 38, or 39 degrees Celsius. Similarly, the controller 214 can activate heating of the system 210 when the outlet temperature signal indicates that a temperature of the perfusate in the circuit 204 is below a threshold, for example 37, 36, or 35 degrees Celsius. In some examples, the controller 214 can modify operation of such components based on an ambient temperature signal from an ambient temperature sensor. The controller 214 can also be configured to produce an alert based on any of the temperature sensor signals.

In another example, the controller 214 can be configured to receive a glucose sensor signal from a glucose sensor (such as of the inlet sensor suite 212a or the outlet sensor suite 212b) and the controller 214 can be configured to operate the pump 206, the gas transfer unit 208, or the injection system 216 based on the glucose sensor signal. For example, if a glucose concentration of the perfusate in the circuit 204 drops below a threshold (for example 0.5 grams per liter), the controller 214 can activate a glucose injection pump 230 to inject glucose into the perfusate circuit 204. The controller 214 can also be configured to produce an alert based on the glucose sensor signal, such as if a glucose consumption rate of the organ 50 drops below a threshold (for example 100 milligrams per hour).

In another example, the controller 214 can be configured to receive a pressure signal from a pressure sensor (such as of the inlet sensor suite 212a or the outlet sensor suite 212b) and the controller 214 can be configured to operate the pump 206 or the cooling system 210 based on the pressure signal. The controller 214 can also be configured to produce an alert based on the pressure signal, such as if the pressure drops below a threshold pressure and the pump is operating, indicating that the pump is failing or has failed. Flow of the pump 206 can be controlled based on monitored pressure of the perfusate circuit 204 (e.g., inlet or outlet of the enclosure 202). The pump 206 can also be controlled to vary a flow rate of perfusate through the perfusate circuit 204, such as between 0 and 2300 milliliters per minute (mm/min). The pump 206 can also be controlled to vary an operating pressure, such as between 0 and 200 millimeters of Mercury (mm/Hg).

In another example, the controller 214 can be configured to receive a gas signal from a gas sensor (such as of the inlet sensor suite 212a or the outlet sensor suite 212b) and can use the gas signal (e.g., O2 or CO2) to determine gas consumption of the organ 50 based on the gas signal. The controller 214 can also operate the gas valves of the gas mixture units 222 to supply a gas mixture to the organ 50 based on the determined gas consumption of the organ. The controller 214 can also operate the injection system 216 based on the gas signal. For example, the controller 214 can adjust a flow rate of the pumps 230 based on consumption of gasses by the organ 50. The controller 214 can also be configured to produce an alert based on the gas signal, such as if the gas levels in the perfusate circuit 204 falls outside an acceptable range or if gas consumption is too high or too low.

Optionally, the tanks of the gas mixture units 222 can each include different blends or concentrations of gasses and the controller 214 can operate one of the gas mixture units 222 (such as 222a) based on the determined concentration of gasses in the perfusate circuit 204 or based on consumption of the organ 50. Alternatively, the tanks of the gas mixture units 222 can all be different gasses and the controller 214 can operate valves of the gas mixture units 222 to achieve a desired inlet blend of gasses into the perfusate circuit 204 based on the concentration of gasses in the perfusate circuit 204 or based on consumption of the organ 50. In either example, the gasses provided by the gas mixture units 222 to the gas transfer unit 208 and the perfusate circuit 204 can be tailored to meet the needs of the organ 50 by the controller 214.

One or more gas meters or flow meters (such as a meter of the flow controller 223 or meters of the gas mixture units 222) can be configured to produce and transmit a gas meter signal to the controller 214. The controller 214 can be configured to operate the gas mixture units 222 or the injection system 216 based on the gas meter signal.

In another example, the controller 214 can be configured to receive a pH signal from a pH sensor (such as of the inlet sensor suite 212a or the outlet sensor suite 212b) and can use the pH signal to determine a pH range of the perfusate in the perfusate circuit 204. The controller 214 can operate one or more of the pumps 230 of the injection system 216 to provide buffer solution (or another fluid) to the perfusate circuit 204 to maintain the pH in a desired or operable range. The controller 214 can also be configured to produce an alert based on the pH signal, such as if the pH levels in the perfusate circuit 204 falls outside an acceptable range. In such an example, the alert may indicate that the organ 50 is contaminated or compromised. The controller 214 can also be configured to produce an alert based on oxygen concentration in the in the media/perfusate. For example, a sharp decrease in the oxygen concentration can be an indicator of contamination.

Optionally, one of the pumps 230 can be a waste pump, operable, such as by the controller 214 to pump fluid (e.g., perfusate) out of the perfusate circuit 204. In such an example, one of the tanks 232 can be a waste tank, which can be emptied or drained manually or automatically. The controller 214 can operate the waste pump to remove media from the perfusate circuit 204 such as when the controller 214 determines, based on one or more sensor signal (e.g., organ health signal or pH signal), that the media is outside a desirable range (e.g., pH) and cannot be recovered or it is not efficient to do so. When such a determination is made, the controller 214 can operate the waste pump to pump media out of the perfusate circuit 204 and into a tank 232 (e.g., 232a). Thereafter, the controller 214 can operate another of the pumps 230 to pump media out of another tank (e.g., 232b) to replenish the media in the perfusate circuit 204. Optionally, the system 200 can include a drain operated by a control valve (operated manually or by the controller 214) to remove waste media or perfusate.

In another example, the controller 214 can be configured to receive the organ health sensor signal from an organ health sensor (such as of the inlet sensor suite 212a or the outlet sensor suite 212b) and the controller 214 can be configured to operate the pump 206 and the gas transfer unit 208 based on the organ health sensor signal. Optionally, the controller 214 can operate the injection system 216 to operate one or more of the pumps 230 to pump a fluid from one or more of the tanks 232 to the perfusate circuit 204. The controller 214 can also be configured to produce an alert based on the organ health sensor signal. In some examples, the organ health sensors can be a metabolite sensor or sensors.

The controller 214 can operate the pumps 230 to inject one or more nutrients to the perfusate circuit 204 to maintain the desired metabolite levels of the organ 50 and the perfusate circuit 204. The organ health sensor can be various types of sensors configured to monitor one or more conditions of the perfusate. For example, the organ health sensor can be an ammonia sensor configured to produce a signal based on an ammonia concentration or level of the perfusate. Also, the organ health sensor can be a glutamine sensor configured to produce a signal based on a glutamine concentration or level of the perfusate. Also, the organ health sensor can be a lactate sensor configured to produce a signal based on a lactate concentration or level of the perfusate. Also, the organ health sensor can be a bile sensor configured to produce a signal based on a bile concentration or level of the perfusate. Also, the organ health sensor can be a creatinine sensor configured to produce a signal based on a creatinine concentration or level of the perfusate. Also, the organ health sensor can be an albumin sensor configured to produce a signal based on an albumin concentration or level of the perfusate. Also, the organ health sensor can be a coagulation time sensor to produce a signal based on an active coagulation time (ACT) of the blood. Clotting factors, such as (Factor VII, Factor VIII, or Factor X), can also be monitored in blood, perfusate, or media. In some examples, the controller 214 can be configured to receive the health signal from the health sensor and can be configured to control an injection of an anticoagulant (such as heparin) if the ACT falls below a threshold (such as below 100 seconds).

In some examples, a sampling port 235 connected to the perfusate circuit 204 (shown in FIG. 2) can be used to take a sample volume out of the perfusate circuit. The sample can be manually tested and the sample can be automatically tested for sampling and analysis during transport. Samples collected from the sampling port 235 can be used by the controller 214 to add biochemical components through the injection system 216 that are necessary to develop and maintain the function of the AT or BEO. Further, automatic sampling of sub-systems can be performed by the controller 214. For example, blood can be automatically drawn from the system and can be delivered to a component that measures ACT, glucose, ammonia, or the like.

In some examples, samples of the blood or media can be taken through the sampling port 235 (or using the glucose sensor 212a). The samples can be assayed (such as by the controller 214) for glucose content of the perfusate over a specified period of time. This can be done in order to determine if the AT or BEO has the adequate glucose consumption rate that meets release specification criteria. When it is determined that the glucose level of the perfusate in the circuit 204 is too low, the controller 214 can operate the injection system 216 to inject glucose into the circuit 204.

In some examples, a specified dose of drugs commonly cleared in the liver can be injected over a specified period of time ((R)-Warfarin, (S)-Mephenytoin, Acetaminophen, Aprepitant, Azole, Benzodiazepines, Beta, Caffeine, Calcium, Carbamazepine, Celecoxib, Clarithromycin, Codeine, Cyclosporine, Delavirdine, Dextromethorphan, Diazepam, Diclofenac, Enalapril, Erythromycin, Estradiol, Estrogen, Fentanyl, Finasteride, Flecainide, Fluoxetine, Glipizide, Glyburide, Haloperidol, Indinavir, Indomethacin, Lidocaine, Lopinavir, Loratidine, Methadone, Mexiletine, Morphine, Nelfinavir, Nifedipine, Olanzapine, Omeprazole, Opioid, Pentamidine, Phenothiazines, Phenytoin, Piroxicam, Prednisone, Progesterone, Propranolol, Quinidine, Risperidone, Ritonavir, Selective serotonin reuptake inhibitors, Saquinavir, Sildenafil, Sirolimus, Statins, Tacrine, Tacrolimus, Tamoxifen, Testosterone, Theophylline, Tramadol, Trazodone, Tricyclic, Valproate, Venlafaxine, Verapamil, or Voriconazole.). Samples of the blood or media can be taken from the sampling port 235 periodically and can be bioanalyzed to determine if the liver BEO (the organ 50) is clearing the drugs to at an adequate rate that meets release criteria. In some examples, samples of the blood or media can be taken from the sampling port 235 and assayed for albumin content over a specified period of time in order to determine if the liver BEO has adequate albumin production that meets release specification criteria. In some examples, samples of the blood or media can be taken from the sampling port 235 and assayed for albumin content over a specified period of time in order to determine if the liver BEO has adequate bile production that meets release specification criteria. In some examples, samples of the blood or media can be taken from the sampling port 235 and can be assayed for creatinine, BSA, and/or urea content over a specified period of time in order to determine if the kidney BEO (the organ 50) has the glomerular filtration rate that meets release specification criteria.

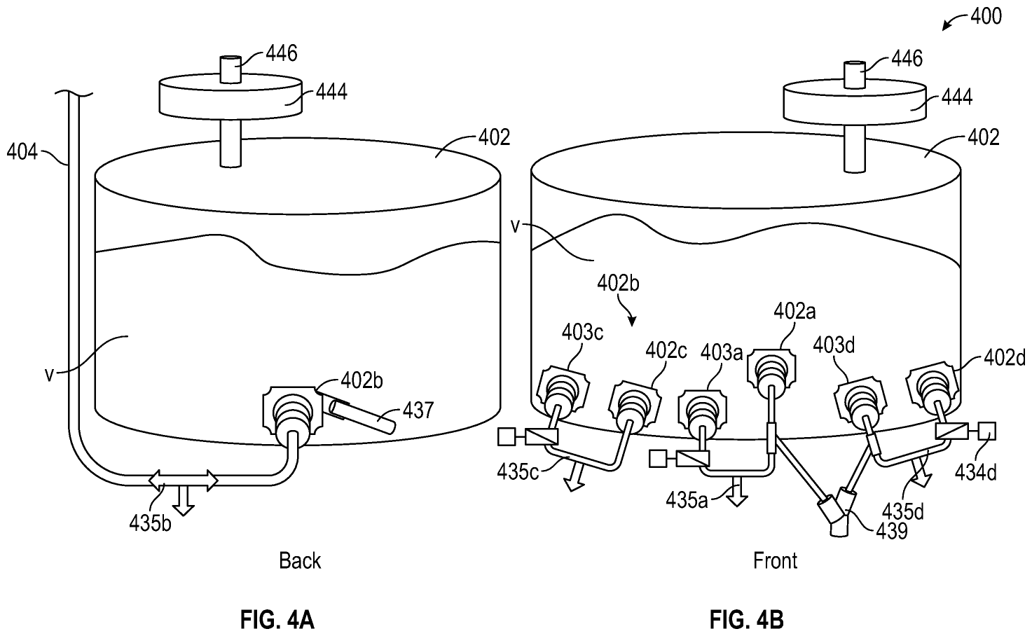

FIG. 4A illustrates a back view of a system 400 for growth and support of a bio-engineered organ. FIG. 4B illustrates a front view of the system 400 for growth and support of a bio-engineered organ. FIGS. 4A and 4B are discussed together below. The system 400 can be similar to the system 200 discussed above and can include all the feature thereof. The system 200 can be modified to include the components of the system 400.

FIG. 4A shows that the system 400 can include multiple connections to an enclosure 402 (similar to the enclosure 202) where the connections can connect directly to chambers or vessels (or sections or portions) of the organ within the enclosure 402. For example, enclosure 402 can include an outlet 402b that can be directly connected to an outlet portion of the organ. The outlet 402b can connect to a perfusate circuit 404 (similar to the circuit 204). The perfusate circuit 404 can include one or more ports 435 that can be a sampling port, dosing port, drain port, or the like. The outlet 402b can connected to a volume V of perfusate or liquid surrounding the organ. Optionally, the outlet 402b can be connected directly to a vessel of the organ 50 in the enclosure 402. Similarly, the outlet 402b can be connected to a port 437 that can be a sampling port, dosing port, drain port, or the like. Such ports can allow for sampling of media within the perfusate circuit 404, draining of the media, or testing of the media.

FIG. 4B shows that the system 400 can include multiple inlet ports 402a and 402d. The system 400 can also include an outlet port 402c. Each of the ports 402a, 402c, and 402d can be connected to a loop including a testing or sampling port 435a, 435c, and 435d, respectively. Each of the loops for each of the ports 402a, 402c, and 402d can also include an isolation control valve (e.g., valve 434d) which can be connected to a controller. The isolation valves can optionally be manual valves.

The port 402a can optionally be a direct connection to an inlet chamber or vessel of the organ. The port 403a can be a connection to the fluid volume V, such as to allow flow into the organ or into the volume. Similarly, the port 402d can optionally be a direct connection to an inlet chamber or vessel of the organ 50 (such as a hepatic duct) and the port 403d can be a connection to the fluid volume V. The port 402c can optionally be a direct connection to an outlet chamber or vessel of the organ 50 (such as the inferior vena cava) and the port 403c can be a connection to the fluid volume V. The system 400 can also include a splitter 439 allowing flow to be introduced into one or more chambers of vessels of the organ 50.

FIGS. 4A and 4B also show that a filter 444 and an outlet 446 can be connected to a lid of the enclosure 402 such as to connect the system 400 to atmospheric pressure to create an open system. The filter 444 can help limit or prevent contamination of the organ 50 and the fluid from the surrounding environment.

In operation of some examples, perfusate can be directed to one or more of the inlets 402a -402d, as controlled by a controller (e.g., the controller 214), such as direct flow to one or more of the chambers or vessels of the organ, which can allow for optimizing perfusion through the organ. Any of the loops of the inlets and outlets can be sampled manually or automatically such as to determine a health or status of the organ or one or more vessels or chambers of the organ.

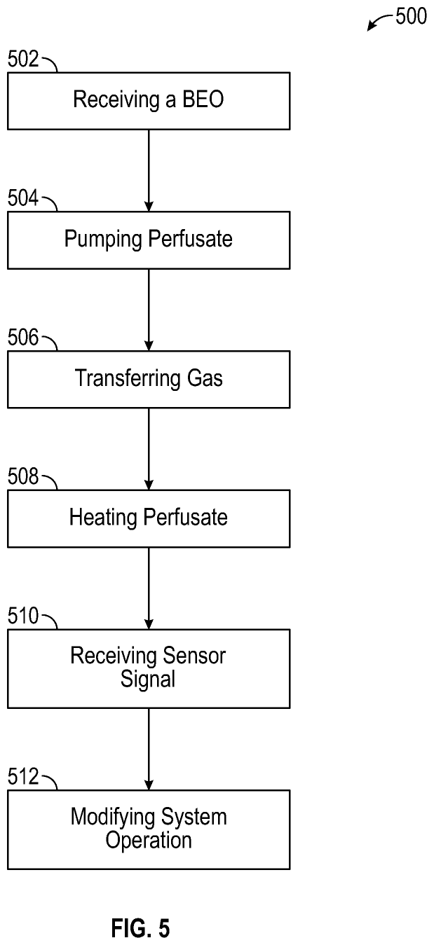

FIG. 5 illustrates a schematic view of a method 500, in accordance with at least one example of the present disclosure. The method 500 can be a method of testing, supporting, and growing a BEO. The steps or operations of the method 500 are illustrated in a particular order for convenience and clarity; many of the discussed operations can be performed in a different sequence or in parallel without materially impacting other operations. The method 500 as discussed includes operations performed by multiple different actors, devices, and/or systems. It is understood that subsets of the operations discussed in the method 500 can be attributable to a single actor, device, or system could be considered a separate standalone process or method.

Method 500 can begin at step 502, where an organ can be received in an enclosure, where the enclosure includes a perfusate inlet and a perfusate outlet. For example, the organ 50 can be received into the enclosure 202. At step 504, perfusate can be pumped through a perfusate circuit connected to the perfusate inlet and the perfusate outlet to circulate perfusate through the system. For example, perfusate can be pumped through the perfusate circuit 204 connected to the perfusate inlet 202a and the perfusate outlet 202b to circulate perfusate through the system 200.

At step 506, gas can be transferred to or from the perfusate using a gas transfer unit connected to the perfusate circuit. For example, gas can be transferred to or from perfusate of the perfusate circuit 204 using the gas transfer unit 208. At step 508, the perfusate can be heated or cooled. Heating or cooling can be controlled by a controller, such as based on a temperature signal of the inlet sensor suite 212a or the outlet sensor suite 212b. At step 510 a sensor signal can be received by the controller. For example, a signal from one or more of the sensors of the inlet sensor suite 212a or the outlet sensor suite 212b can be received by the controller 214. At step 512 operation of the system can be modified based on the signal. For example, the controller 214 can operate one or more components of the system 200 based on the one or more signals, as discussed above with respect to FIGS. 2 and 3.

In some examples the injection system (e.g., the injection system 216) can inject one or more nutrients into the system via the perfusion circuit. In another example, a sensor signal can be produced based on a condition of the perfusate. For example, the inlet sensor suite 212a or the outlet sensor suite 212b can produce a sensor signal. Optionally, the pump and the injection system can be operated based on the sensor signal. For example, the controller 214 can operate the pump 206 or the injection system 216 based on the signal.

In another example, a gas mixture unit can be operated to deliver gas to the gas transfer unit. For example, the gas mixture units 222 can be operated to deliver gas to the gas transfer unit 208. Optionally, a plurality of gas mixture units (e.g., the gas mixture units 222) connected to the gas transfer unit can be configured to provide different gasses or different gas concentrations to the gas transfer unit based on the sensor signal. In some examples, a plurality of gas control valves can each connected to one of the gas mixture units gas mixture units and each in communication with the controller, and the controller can be configured to operate the plurality of gas control valves to deliver gasses to the gas transfer unit based on the sensor signal. In some examples, the gas mixture unit can deliver one or more of Carbon Dioxide, Dioxygen, Nitrogen, or Argon to the gas transfer unit based on the sensor signal.

In another example, the sensor can be a plurality of gas sensors each configured to transmit a gas signal to the controller and the controller can be configured to determine gas consumption of the organ based on the sensor signals, and the controller can be configured to operate the plurality of gas control valves to supply a gas mixture to the organ based on the determined gas consumption of the organ.

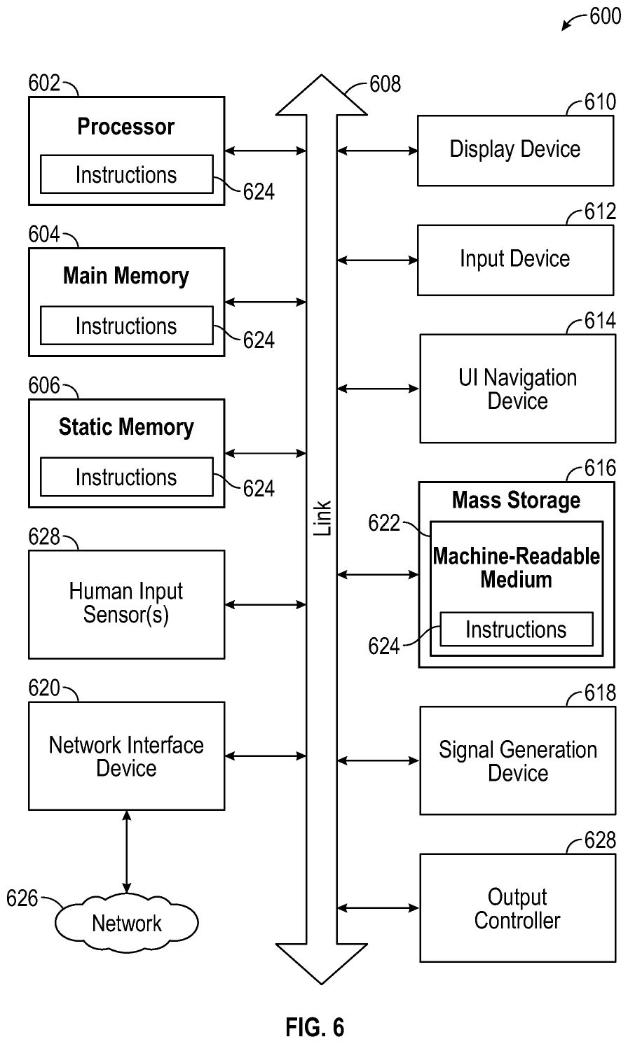

FIG. 6 illustrates a block diagram of an example machine 600 upon which any one or more of the techniques (e.g., methodologies) discussed herein can perform. Examples, as described herein, can include, or can operate by, logic or a number of components, or mechanisms in the machine 600 (which can be the system 200). Circuitry (e.g., processing circuitry) is a collection of circuits implemented in tangible entities of the machine 600 that include hardware (e.g., simple circuits, gates, logic, etc.). Circuitry membership can be flexible over time. Circuitries include members that may, alone or in combination, perform specified operations when operating. In an example, hardware of the circuitry can be immutably designed to carry out a specific operation (e.g., hardwired). In an example, the hardware of the circuitry can include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components can be used in more than one member of more than one circuitry. For example, under operation, execution units can be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 can operate as a standalone device or can be connected (e.g., networked) to other machines. In a networked deployment, the machine 600 can operate in the capacity of a server machine, a client machine, or both in server-client network environments. In an example, the machine 600 can act as a peer machine in peer-to-peer (P2P) (or other distributed) network environment. The machine 600 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 600 can include a hardware processor 602 (e.g., a central processing unit (CPU), a graphics processing unit (GPU), a hardware processor core, or any combination thereof), a main memory 604, a static memory (e.g., memory or storage for firmware, microcode, a basic-input-output (BIOS), unified extensible firmware interface (UEFI), etc.) 606, and mass storage 608 (e.g., hard drive, tape drive, flash storage, or other block devices) some or all of which can communicate with each other via an interlink (e.g., bus) 630. The machine 600 can further include a display unit 610, an alphanumeric input device 612 (e.g., a keyboard), and a user interface (UI) navigation device 614 (e.g., a mouse). In an example, the display unit 610, input device 612 and UI navigation device 614 can be a touch screen display. The machine 600 can additionally include a storage device (e.g., drive unit) 608, a signal generation device 618 (e.g., a speaker), a network interface device 620, and one or more sensors 616, such as a global positioning system (GPS) sensor, compass, accelerometer, or other sensor. The machine 600 can include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 602, the main memory 604, the static memory 606, or the mass storage 608 can be, or include, a machine readable medium 622 on which is stored one or more sets of data structures or instructions 624 (e.g., software) embodying or utilized by any one or more of the techniques or functions described herein. The instructions 624 can also reside, completely or at least partially, within any of registers of the processor 602, the main memory 604, the static memory 606, or the mass storage 608 during execution thereof by the machine 600. In an example, one or any combination of the hardware processor 602, the main memory 604, the static memory 606, or the mass storage 608 can constitute the machine readable media 622. While the machine readable medium 622 is illustrated as a single medium, the term “machine readable medium” can include a single medium or multiple media (e.g., a centralized or distributed database, and/or associated caches and servers) configured to store the one or more instructions 624.

The term “machine readable medium” can include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine readable medium examples can include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media can include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 can be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 can include one or more physical jacks (e.g., Ethernet, coaxial, or phone jacks) or one or more antennas to connect to the communications network 626. In an example, the network interface device 620 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software. A transmission medium is a machine readable medium.

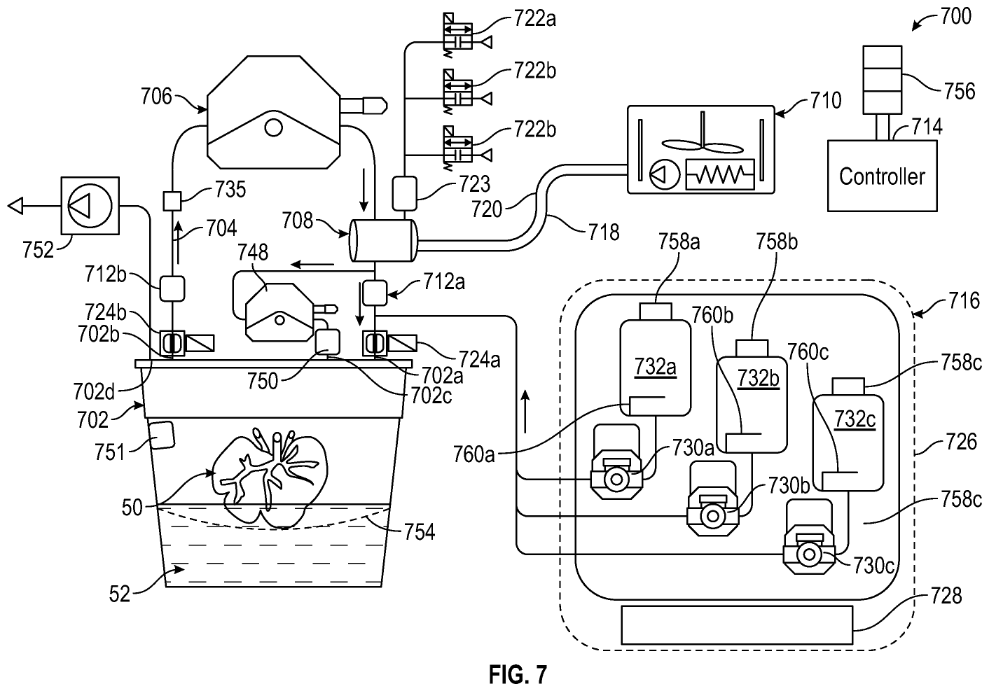

FIG. 7 illustrates a schematic view of a system 700 for growth and support of a bio-engineered organ. The system 700 can be similar to the system 200 discussed above; the system 700 can include additional features or components, such as a vacuum pump and a secondary pump. Any of the systems discussed above or below can be modified to include the features of the system 700. In the system 700, components with reference numerals similar to those of the system 200 can reference similar components that can be connected and can operate as described above.

More specifically, the system 700 can include a secondary pump 748 that can be connected to a circuit 704 and can be configured to circulate perfusate through the perfusate circuit 704. The secondary pump 748 can be a positive displacement pump, a centrifugal pump, or an axial pump. The secondary pump 748 can be a low-shear pump to help limit damage to the fluid and can optionally be reversible. In some examples, the pump 748 can be a continuous-type pump or a peristaltic type pump (with or without damping). In some examples, the secondary pump 748 can be located downstream of a primary pump 706 and upstream of an enclosure 702.

The system 700 can also include a secondary inlet sensor suite 750 located between a discharge of the secondary pump 748 and a secondary inlet 702c. The secondary inlet sensor suite 750 can optionally include one or more of a pressure sensor, a temperature sensor, a flow meter, a glucose sensor, an O2 sensor, a CO2 sensor, or other sensors. Each sensor can be connected to the perfusate circuit 704 upstream of the enclosure 702 (and can be optionally be within the enclosure 702). The secondary inlet sensor suite 750 can be configured to transmit one or more signals to the controller 714.

The system 700 can also include an enclosure sensor suite 751 located at least partially within the enclosure 702 and connected to the controller 714. The enclosure sensor suite 751 can optionally include one or more of a pressure sensor, a temperature sensor, a flow meter, a glucose sensor, an O2 sensor, a CO2 sensor, or other sensors.

The secondary pump 748 can be connected to and in communication with a controller 714 such that the controller 714 can control operation of the pump 748 based on one or more sensor signals of the system 700 or one or more determinations made using the signals. Similarly, the secondary inlet sensor 750 can be connected to and in communication with the controller 714. The controller 714 can be configured to operate one or more of the primary pump 706 and the secondary pump 748 based on the one or more signals from the secondary inlet sensor suite 750 or any other sensor of the system 700.

In operation of the system 700, the primary pump 706 can be configured to provide or deliver fluid (such as perfusate or blood) through an inlet 702a which can be connected (e.g., directly) to one or more primary circuits or vessels of the organ 50. The controller 714 can control operation of the primary pump 706 to deliver perfusate to the inlet 702a at a pressure or flowrate to properly perfuse the primary circuits of the organ 50. Meanwhile, the pump 748 can be configured to deliver fluid to one or more secondary circuits or vessels of the organ 50, which may require different operating pressures or flow rates than the primary vessels. The controller 714 can control operation of the pump 748 to deliver perfusate to the inlet 702c at a pressure or flowrate to properly perfuse the secondary vessel(s) of the organ 50. In this way, the system 700 can have flow distribution flexibility or variability to improve organ health or growth.

FIG. 7 also shows that the system 700 can include a vacuum pump 752 that can be connected to an outlet or vent 702d. The vacuum pump 752 can be connected to the controller 714 such that the controller 714 can operate the vacuum pump 752 configured to create a vacuum in the air (or gas) pressure in the enclosure 702. Creating an environment with a negative pressure (or lower pressure) relative to atmospheric pressure can help to promote perfusion of perfusate and cells through the organ, such as through an outer portion thereof, which can help to improve organ health or growth of a biologically engineered organ.

FIG. 7 also shows that the enclosure 702 can include a support 754 that can be connected to one or more wall of the enclosure 702. The support 754 can support or suspend the organ 50 within the enclosure 702 to help maintain organ shape and function during growth, decellularization, or recellularization of the organ 50.

Finally, FIG. 7 shows that the system 700 can include an indicator 756. The indicator 756 can be a series of lights or other visual indicator(s) connected to the controller 714. The indicator 756 can be configured to receive a signal from the controller 714 to control illuminating or enabling one or more of the lights (e.g., red, yellow, or green) based on one or more signals of the system 700 or based on one or more determinations made using the signal(s).

FIG. 7 also shows that the system 700 can include force sensors 758a-758c. The force sensor 758a can be connected to a tank 732a, the force sensor 758c can be connected to a tank 732b, and the force sensor 758c can be connected to a tank 732c. The force sensors 758 can be scales or other forces sensors configured produce a signal based on a force applied by the tank, such as to correlate to a weight of each tank. The force sensors 758 can be connected to and in communication with the controller 714 such as to transmit the force signals thereto. For example, the force sensor 758a can produce a force signal based on a weight or mass of the tank, which can be transmitted to the controller 714 for determination of a weight of fluid within the tank 732a. The controller 714 can use the force signal to determine a change in volume or weight of fluid within the tank 732a, allowing the 714 to control the pump 730 to inject a fluid from the tank 732a based on mass, weight, or volume. In this way, the controller 714 can operate the pumps 730 inject nutrients into the circuit 704 by weight or mass.

The system 700 can also include agitation devices 760a-760c connected to respective tanks 732a-732c. The agitation devices 760a-760c can be located within respective tanks 732a-732c, or can be located in the circuit downstream or upstream of respective pumps 730a-730c. Each mixer 760 can include a motor in communication with the controller 714, such that the controller 714 can operate one or more agitation device 760a-760c to mix contents within the tanks 732 or as the contents are pump therefrom. The agitation devices 760a-760c can thereby help to promote uniform distribution of solutions, which can be important for cell solutions that settle out or separate.

Also, an inlet sensor suite 712a can include a bubble sensors configured to produce a bubble signal based on detection of one or more bubbles within the circuit 704, such as upstream of the inlet 702a. The inlet sensor suite 712a can be in communication with the controller 714 such as to transmit the bubble signal thereto to allow the controller 714 to detect the presence of a bubble within the circuit 704. Upon detection of a bubble in the circuit 704, the controller 714 can operate the primary pump 706, such as stopping the pump, to help limit bubble(s) from entering the organ 50 (AT or BEO), and helping to limit damage to the organ 50.

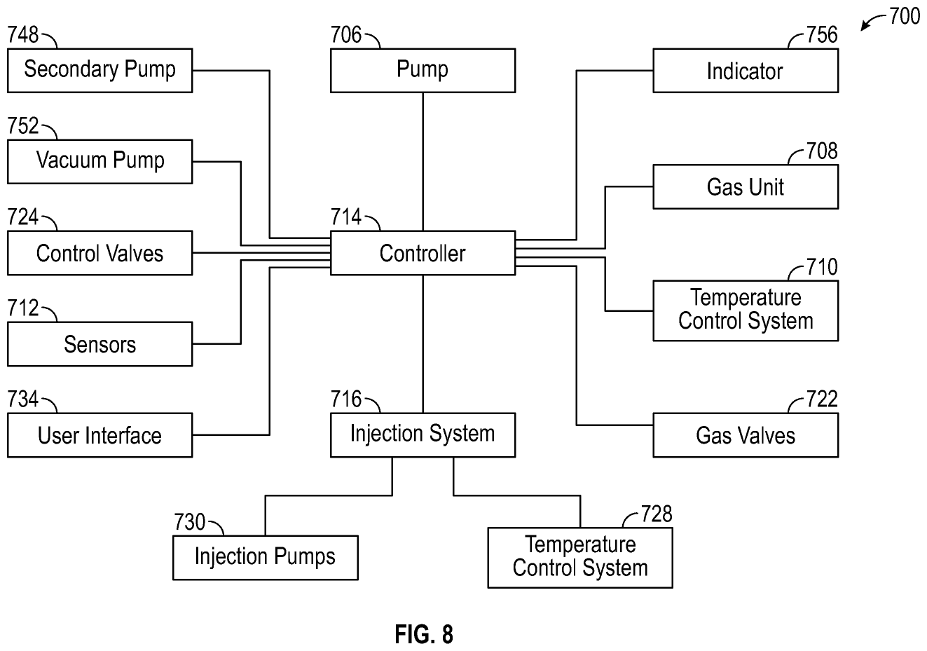

FIG. 8 illustrates a schematic view of a system 700 for growth and support of a bio-engineered organ. The system 700 can be similar to the system 200 discussed above; the system 700 can include additional features or components, such as a vacuum pump and a secondary pump. Any of the systems discussed above or below can be modified to include the features of the system 700. In the system 700, components with reference numerals similar to those of the system 200 can reference similar components that can be connected and can operate as described above.

As discussed above with reference to FIG. 7, the system 700 can include a secondary pump 748 and a vacuum pump 752 that can be connected to the controller 714. The system 700 can also include the indicator 756 that can be connected to the controller 714. The secondary pump 748 can be operated by the controller 714 to control a flow of perfusate into the secondary inlet 702c, such as by controlling a flow rate or pressure of perfusate into the secondary inlet 702c. For example, the controller 714 can operate the pump 748 to control a flow rate or pressure of perfusate into the secondary inlet 702c based on one or more signals from the secondary inlet sensor suite 750, such as a pressure sensor signal or a flow rate signal. The controller 714 can control flowrate and pressure of the perfusate based on optimal growth or health of the organ 50. For example, the pump 748 can be controlled to deliver a flow, for example between 0 and 100 ml/min at a pressure between 0 and 100 mm Hg.

The vacuum pump 752 can be operated by the controller 714 to control a gas pressure, air pressure, or ambient pressure within the container relative to the perfusate 52. For example, the controller 714 can operate the vacuum pump 752 to control a pressure of gas within the enclosure 702 based on one or more signals from the enclosure sensor suite 751, such as a pressure sensor signal. The controller 714 can control the vacuum pump 752 to maintain a pressure within the enclosure 702 to optimize growth or health of the organ 50. For example, the secondary inlet sensor suite 750 can be controlled to maintain a pressure within the enclosure 702, such as between 0 and −50 mm Hg of vacuum gauge pressure.

The indicator 756 can include one or more visual indicators such as lights emitting various colors. For example, the indicator 756 can include a red light, a yellow light, and a green light. The controller 714 can control each of the lights based on one or more signals from the system 700. For example, the controller 714 can enable the green light when the system 700 is operating normally. The controller 714 can enable the yellow light when the system 700 is operating abnormally and requires attention. The controller 714 can enable the red light when the system 700 is operating sub-optimally or requires immediate attention or action. Optionally, the lights can be controlled to flash or blink, such as based on one or more sensor signals of the system 700 or based on one or more determinations made by the controller 714 based on the one or more signals of the system 700.

### Notes and Examples

The following, non-limiting examples, detail certain aspects of the

present subject matter to solve the challenges and provide the benefits discussed herein, among others.

Example 1 is a system for growing or supporting an organ, the system comprising: an enclosure configured to support the organ therein in a perfusate flow, the enclosure including a perfusate inlet and a perfusate outlet to receive the perfusate flow through the enclosure and through the organ; a perfusate circuit connected to the perfusate inlet and the perfusate outlet and configured to circulate perfusate through the system and the organ; a perfusion pump connected to the circuit and configured to circulate perfusate through the perfusate circuit; a gas transfer unit connected to the perfusate circuit and configured to transfer gas to and from the perfusate; a sensor connected to the perfusate circuit and configured to produce a sensor signal based on a condition of the perfusate; an injection system connected to the perfusion circuit, the injection system configured to inject nutrients into the system; and a controller configured to operate the pump, the gas transfer unit, and the injection system based on the sensor signal.

In Example 2, the subject matter of Example 1 optionally includes a gas mixture unit connected to the gas transfer unit and configured to deliver gas to the gas transfer unit.

In Example 3, the subject matter of Example 2 optionally includes wherein the gas mixture unit delivers one or more of Carbon Dioxide, Dioxygen, Nitrogen, or Argon to the gas transfer unit.

In Example 4, the subject matter of Example 3 optionally includes a gas control valve connected to the gas mixture unit and in communication with the controller, the controller configured to operate the control valve to deliver the gas to the gas transfer unit based on the sensor signal.

In Example 5, the subject matter of Example 4 optionally includes wherein the sensor is a gas sensor configured to transmit a gas signal to the controller, the controller is configured to determine gas consumption of the organ based on the sensor signal, and wherein the controller is configured to operate the gas valve to supply a gas mixture to the organ based on the determined gas consumption of the organ.

In Example 6, the subject matter of Example 5 optionally includes a gas meter connected to the gas mixture unit and configured to transmit a gas meter signal to the controller, the controller configured to operate the control valve based on the gas meter signal.

In Example 7, the subject matter of any one or more of Examples 3-6 optionally include a plurality of gas mixture units connected to the gas transfer unit and configured to different gasses or different gas concentrations to the gas transfer unit.

In Example 8, the subject matter of Example 7 optionally includes a plurality of gas control valves each connected to one of the gas mixture units gas mixture units and each in communication with the controller, the controller configured to operate the plurality of gas control valves to deliver gasses to the gas transfer unit based on the sensor signal.

In Example 9, the subject matter of Example 8 optionally includes wherein the sensor is a plurality of gas sensors each configured to transmit a gas signal to the controller, the controller is configured to determine gas consumption of the organ based on the sensor signals, and wherein the controller is configured to operate the plurality of gas control valves to supply a gas mixture to the organ based on the determined gas consumption of the organ.

In Example 10, the subject matter of any one or more of Examples 3-9 optionally include wherein the gas transfer unit is an oxygenator.

In Example 11, the subject matter of Example 10 optionally includes wherein the gas transfer unit includes an air separator.

In Example 12, the subject matter of any one or more of Examples 2-11 optionally include a heating system connected to the gas transfer unit to exchange heat with the perfusate through the gas transfer unit.

In Example 13, the subject matter of Example 12 optionally includes an inlet temperature sensor connected to the circuit upstream of the enclosure, the inlet temperature sensor configured to produce an inlet temperature signal based on an inlet temperature of the perfusate entering the enclosure; and an outlet temperature sensor connected to the circuit downstream of the enclosure, the outlet temperature sensor configured to produce an outlet temperature signal based on an outlet temperature of the perfusate leaving the enclosure.

In Example 14, the subject matter of Example 13 optionally includes wherein the controller is configured to: receive the inlet temperature signal and the outlet temperature signal; and operate the pump, the gas transfer unit, and the heating system based on the inlet temperature signal and the outlet temperature signal.

In Example 15, the subject matter of any one or more of Examples 1-14 optionally include an injection system connected to the perfusate circuit upstream of the enclosure and in communication with the controller, the controller configured to operate the injection system based on the sensor signal to deliver supplements to the perfusate.

In Example 16, the subject matter of Example 15 optionally includes wherein the injection system includes a plurality of injection pumps each configured to deliver a supplement to the perfusate, the controller configured to operate each of the injection pumps based on the sensor signal to deliver supplements to the perfusate.

In Example 17, the subject matter of Example 16 optionally wherein the plurality of injection pumps are each configured to deliver to the perfusate and the organ, one or more of cell culture media, cells, glutamine, glucose, buffer, sodium chloride, essential amino acids, non-essential amino acids, drugs, ammonia (ammonium chloride), HEPES (C₈H₁₈N₂O₄S), Sodium Bicarbonate, Insulin, Epinephrine, Albumin, linoleic acid, dexamethasone, and glucagon.

In Example 18, the subject matter of any one or more of Examples 16-17 optionally include wherein the injection system includes an enclosure supporting the plurality of injection pumps and the supplements.

In Example 19, the subject matter of Example 18 optionally includes wherein the injection system includes a cooling system configured to cool an environment of the injection system, the cooling system in communication with the controller, the controller configured to operate the cooling system to maintain a desired temperature of the environment of the injection system.

In Example 20, the subject matter of any one or more of Examples 18-19 optionally include wherein the injection system includes a cooling system configured to cool an environment of the injection system, the cooling system in communication with the controller, the controller configured to operate the cooling system to maintain a desired temperature of the environment of the injection system.

In Example 21, the subject matter of Example 20 optionally includes an inlet control valve connected to the perfusate circuit at the inlet of the enclosure, the inlet control valve in communication with the controller; and an outlet control valve connected to the perfusate circuit at the outlet of the enclosure, the outlet control valve in communication with the controller and the controller configured to operate the inlet control valve and the outlet control valve to isolate the enclosure and the organ.

In Example 22, the subject matter of any one or more of Examples 1-21 optionally include a secondary perfusate pump connected to the circuit downstream of the perfusate pump and configured to circulate perfusate through a secondary vessel of the organ.

In Example 23, the subject matter of any one or more of Examples 1-22 optionally include a vacuum pump connected to the enclosure and configured to create a negative air pressure within the enclosure.

Example 24 is a method for growing or supporting a organ using a system, the method comprising: receiving a organ in an enclosure, the enclosure including a perfusate inlet and a perfusate outlet; pumping perfusate through a perfusate circuit connected to the perfusate inlet and the perfusate outlet to circulate perfusate through the system; transferring gas to or from the perfusate using a gas transfer unit connected to the perfusate circuit; injecting nutrients into the system using an injection system connected to the perfusion circuit.

In Example 25, the subject matter of Example 24 optionally includes producing a sensor signal based on a condition of the perfusate; and operating the pump and the injection system based on the sensor signal.

In Example 26, the subject matter of Example 25 optionally includes operating a gas mixture unit to deliver gas to the gas transfer unit.

In Example 27, the subject matter of Example 26 optionally includes wherein the gas mixture unit delivers one or more of Carbon Dioxide, Dioxygen, Nitrogen, or Argon to the gas transfer unit based on the sensor signal.

In Example 28, the subject matter of Example 27 optionally includes operating a plurality of gas mixture units connected to the gas transfer unit and configured to provide different gasses or different gas concentrations to the gas transfer unit based on the sensor signal.

In Example 29, the subject matter of Example 28 optionally includes operating a plurality of gas control valves each connected to one of the gas mixture units gas mixture units and each in communication with the controller, the controller configured to operate the plurality of gas control valves to deliver gasses to the gas transfer unit based on the sensor signal.

In Example 30, the subject matter of Example 29 optionally includes wherein the sensor is a plurality of gas sensors each configured to transmit a gas signal to the controller, the controller is configured to determine gas consumption of the organ based on the sensor signals, and wherein the controller is configured to operate the plurality of gas control valves to supply a gas mixture to the organ based on the determined gas consumption of the organ.

Example 31 is an apparatus comprising means to implement of any of Examples 1-29.

Example 32 is a system to implement of any of Examples 1-29.

Example 33 is a method to implement of any of Examples 1-29.

In Example 34, the apparatuses or method of any one or any combination of Examples 1-33 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system for growing or supporting an organ, the system comprising:
an enclosure configured to support the organ therein in a perfusate flow, the enclosure including a perfusate inlet and a perfusate outlet to receive the perfusate flow through the enclosure and through the organ;
a perfusate circuit connected to the perfusate inlet and the perfusate outlet and configured to circulate perfusate through the system and the organ;
a perfusion pump connected to the circuit and configured to circulate perfusate through the perfusate circuit;
a gas transfer unit connected to the perfusate circuit and configured to transfer gas to and from the perfusate;
a sensor connected to the perfusate circuit and configured to produce a sensor signal based on a condition of the perfusate;
an injection system connected to the perfusion circuit, the injection system configured to inject nutrients into the system; and
a controller configured to operate the pump, the gas transfer unit, and the injection system based on the sensor signal.

2. The system of claim 1, further comprising:
a gas mixture unit connected to the gas transfer unit and configured to deliver gas to the gas transfer unit.

3. The system of claim 2, wherein the gas mixture unit delivers one or more of Carbon Dioxide, Dioxygen, Nitrogen, or Argon to the gas transfer unit.

4. The system of claim 3, further comprising:
a gas control valve connected to the gas mixture unit and in communication with the controller, the controller configured to operate the control valve to deliver the gas to the gas transfer unit based on the sensor signal.

5. The system of claim 4, wherein the sensor is a gas sensor configured to transmit a gas signal to the controller, the controller is configured to determine gas consumption of the organ based on the sensor signal, and wherein the controller is configured to operate the gas valve to supply a gas mixture to the organ based on the determined gas consumption of the organ.

6. The system of claim 5, further comprising:
a gas meter connected to the gas mixture unit and configured to transmit a gas meter signal to the controller, the controller configured to operate the control valve based on the gas meter signal.

7. The system of claim 3, further comprising:
a plurality of gas mixture units connected to the gas transfer unit and configured to different gasses or different gas concentrations to the gas transfer unit.

8. The system of claim 7, further comprising:
a plurality of gas control valves each connected to one of the gas mixture units gas mixture units and each in communication with the controller, the controller configured to operate the plurality of gas control valves to deliver gasses to the gas transfer unit based on the sensor signal.

9. The system of claim 8, wherein the sensor is a plurality of gas sensors each configured to transmit a gas signal to the controller, the controller is configured to determine gas consumption of the organ based on the sensor signals, and wherein the controller is configured to operate the plurality of gas control valves to supply a gas mixture to the organ based on the determined gas consumption of the organ.

10. The system of claim 3, wherein the gas transfer unit is an oxygenator.

11. The system of claim 10, wherein the gas transfer unit includes an air separator.

12. The system of claim 2, further comprising:
a heating system connected to the gas transfer unit to exchange heat with the perfusate through the gas transfer unit.

13. The system of claim 12, further comprising:
an inlet temperature sensor connected to the circuit upstream of the enclosure, the inlet temperature sensor configured to produce an inlet temperature signal based on an inlet temperature of the perfusate entering the enclosure; and
an outlet temperature sensor connected to the circuit downstream of the enclosure, the outlet temperature sensor configured to produce an outlet temperature signal based on an outlet temperature of the perfusate leaving the enclosure.

14. The system of claim 13, wherein the controller is configured to:
receive the inlet temperature signal and the outlet temperature signal; and
operate the pump, the gas transfer unit, and the heating system based on the inlet temperature signal and the outlet temperature signal.

15. The system of claim 1, further comprising:
an injection system connected to the perfusate circuit upstream of the enclosure and in communication with the controller, the controller configured to operate the injection system based on the sensor signal to deliver supplements to the perfusate.

16. The system of claim 15, wherein the injection system includes a plurality of injection pumps each configured to deliver a supplement to the perfusate, the controller configured to operate each of the injection pumps based on the sensor signal to deliver supplements to the perfusate.

17. The system of claim 16, wherein the plurality of injection pumps are each configured to deliver to the perfusate and the organ, one or more of cell culture media, cells, glutamine, glucose, buffer, sodium chloride, essential amino acids, non-essential amino acids, drugs, ammonia (ammonium chloride), HEPES (C₈H₁₈N₂O₄S), Sodium Bicarbonate, Insulin, Epinephrine, Albumin, linoleic acid, dexamethasone, and glucagon.

18. The system of claim 16, wherein the injection system includes an enclosure supporting the plurality of injection pumps and the supplements.

19. The system of claim 18, wherein the injection system includes a cooling system configured to cool an environment of the injection system, the cooling system in communication with the controller, the controller configured to operate the cooling system to maintain a desired temperature of the environment of the injection system.

20. The system of claim 19, further comprising:
an inlet control valve connected to the perfusate circuit at the inlet of the enclosure, the inlet control valve in communication with the controller; and
an outlet control valve connected to the perfusate circuit at the outlet of the enclosure, the outlet control valve in communication with the controller and the controller configured to operate the inlet control valve and the outlet control valve to isolate the enclosure and the organ.

21. The system of claim 1, further comprising:
a secondary perfusate pump connected to the circuit downstream of the perfusate pump and configured to circulate perfusate through a secondary vessel of the organ.

22. The system of claim 1, further comprising:
a vacuum pump connected to the enclosure and configured to create a negative air pressure within the enclosure.

23-29. (canceled)
